(19) United States
(12) Patent Application Publication
(10) Pub. No.: US 2026/0294504 A1
(43) Pub. Date: Oct. 1, 2026

(54) DEVICES AND METHODS FOR MUCOSAL SURFACE REMODELING

(71) Applicant: Aerin Medical Inc., Mountain View, CA (US)

(72) Inventor: Scott J. Wolf, Menlo Park, CA (US)

(73) Assignee: Aerin Medical Inc., Mountain View, CA (US)

(21) Appl. No.: 19/636,824

(22) Filed: Apr. 1, 2026

Related U.S. Application Data
(60) Provisional application No. 63/781,706, filed on Apr. 1, 2025.

(51) Int. Cl. A61B 18/02; A61B 18/00

(52) U.S. Cl. CPC A61B 18/0218; A61B 2018/00577

(57) ABSTRACT

Systems and methods are provided for treating tissue in a nasal airway. In some embodiments, a method of resurfacing a mucosal layer is provided that includes applying a stimulus to an existing mucosal tissue to a predetermined depth, the predetermined depth being a depth within a mucosal layer. The stimulus treats the existing mucosal tissue along the predetermined depth to cause replacement of the existing mucosal tissue with healthy mucosal tissue, thereby resurfacing a surface of the mucosal layer.

### RELATED APPLICATIONS

This application claims the benefit of and priority to U.S. Provisional Application No. 63/781,706, filed Apr. 1, 2025, the contents of which is hereby incorporated herein by reference in its entirety.

### FIELD

This disclosure relates methods and systems for remodeling and resurfacing tissue.

### BACKGROUND

Rhinitis is generally the inflammation of the nasal mucosa due to allergic or non-allergic triggers which leads to excessive mucus production in the nasal cavity. This increased mucus can cause runny nose, post nasal drip syndrome (PNDS), upper airway cough syndrome (UACS), congestion, itching and sneezing symptoms. Chronic Rhinitis is a chronic nasal condition that affects millions of people. There is still a need for an efficient, non-invasive treatment of these conditions.

### SUMMARY

The present disclosure relates to systems and methods that trigger or stimulate mucosal remodeling through direct stimulus from an agent or a stimulus. The resurfacing of the mucosa replaces the diseased or chronically inflamed mucosal cells with healthy mucosa that has more normal function, such as producing less mucus or other fluids, being less prone to allergy, and having less nerve stimulation, fewer nerve endings or receptors, thinner mucosal layer, less activity of inflammatory cells, or less inflammatory cells. In some embodiments, this can be through modification of the cell types that comprise the mucosal layers.

Accordingly, in some embodiments the present disclosure provides a method of resurfacing a mucosal layer, the method comprising: advancing a treatment device into a nasal cavity; applying a stimulus to a mucosal surface to a predetermined depth, wherein the predetermined depth is selected to resurface the mucosal surface and wherein the stimulus comprises cryo-energy; and removing the device from the nasal cavity.

In some embodiments, a method of resurfacing a mucosal layer is provided that includes applying a stimulus to an existing mucosal tissue to a predetermined depth, the predetermined depth being a depth within a mucosal layer, wherein the stimulus treats the existing mucosal tissue along the predetermined depth to cause replacement of the existing mucosal tissue with new mucosal tissue, thereby resurfacing a surface of the mucosal layer.

In some embodiments, the predetermined depth extends to a distal end of nerve endings in the existing mucosal tissue. In some embodiments the predetermined depth is less than a depth of the mucosal layer. In some embodiments, the predetermined depth is 3.5 mm.

In some embodiments the stimulus includes a cryoablation spray. In In some embodiments the stimulus is radiofrequency energy. In some embodiments the stimulus is pulsed field ablation.

In some embodiments, the resurfacing of the surface of the existing mucosal tissue causes fewer mucous glands, fewer mucous producing cells, less inflammation, less inflammatory cells, thinner mucosal layer, or more normal nerve function.

In some embodiments, a method of resurfacing a mucosal layer is provided that includes advancing a treatment device into a nasal cavity; controlling, with a control system, a depth and amount of a stimulus being applied to mucosal tissue in the nasal cavity; applying the stimulus with the treatment device to the mucosal tissue to a depth selected by the control system, wherein the depth is selected allow the stimulus to treat the mucosal tissue to resurface a surface of the mucosal tissue; and removing the treatment device from the nasal cavity.

In some embodiments, the stimulus treats the mucosal tissue along the depth to cause replacement of the mucosal tissue with healthy mucosal tissue, thereby resurfacing a surface of the mucosal layer.

In some embodiments, the depth extends to a distal end of nerve endings in the mucosal tissue. In some embodiments, the depth is less than a depth of the mucosal layer. In some embodiments, the depth is 3.5 mm.

In some embodiments, a method of resurfacing a mucosal layer is provided that includes advancing a treatment device into a nasal cavity, the treatment device including: a treatment element having one or more ports for delivering a stimulus in the form of a cryoablation spray; a stimulus supply in fluid communication with the one or more ports of the treatment element, a control system configured to control an amount of stimulus delivered from the treatment element; applying power, with a power supply, to drive an amount of the stimulus from the stimulus supply to the treatment element to apply the stimulus to treat existing mucosal tissue along a predetermined depth, the predetermined depth being a depth within a mucosal layer, wherein the predetermined depth is selected to allow the stimulus treat the existing mucosal tissue along the predetermined depth to cause replacement of the existing mucosal tissue with healthy mucosal tissue, thereby resurfacing a surface of the mucosal layer; and removing the treatment device from the nasal cavity.

In some embodiments, the resurfacing of the surface of the existing mucosal tissue causes fewer mucous glands, fewer mucous producing cells, less inflammation, less inflammatory cells, thinner mucosal layer, or more normal nerve function.

In some embodiments, applying the stimulus causes one or more changes to properties of the mucosal layer.

In some embodiments, the predetermined depth extends to a distal end of nerve endings in the mucosal layer, without disrupting the nerves. In some embodiments, the predetermined depth is less than a depth of the mucosal layer. In some embodiments, the predetermined depth is 3.5 mm.

In some embodiments, the treatment element includes one or more thermocouples for providing temperature feedback of or relating to the mucosal tissue, the temperature feedback allowing for adjusting one or more variables of delivery of the cryoablation spray, including a flow rate of cryoablation spray to the one or more ports of the treatment element or a flow rate of the cryoablation spray out of the one or more ports of the treatment element.

### BRIEF DESCRIPTION OF THE DRAWINGS

The following presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIGS. 1A and 1B show an exemplary cross-sectional view of mucosa, according to one or more embodiments herein;

FIG. 2 shows a top view of a mucosa treatment device, according to one or more embodiments herein;

FIG. 3 shows a side view of an exemplary cryospray dispenser as a treatment element, according to one or more embodiments herein; and

FIG. 4A shows a side view of an exemplary balloon as a treatment element of a treatment device, according to one or more embodiments herein; and

FIG. 4B shows a perspective view of a balloon treatment element including one or more energy transfer elements positioned thereon, according to one or more embodiments herein;

FIG. 5 shows a perspective view of a vacuum cooling member as a treatment element, according to one or more embodiments herein;

FIG. 6 shows a flowchart of an exemplary method of remodeling or resurfacing mucosa, according to one or more embodiments herein;

FIG. 7A illustrates an exemplary example method of use of the various devices disclosed herein;

FIG. 7B is a sagittal view of a human nasal cavity showing an exemplary embodiment of multiple treatments performed on nasal tissue with a multichannel device; and

FIGS. 8A and 8B show an exemplary cross-sectional view of the nasal cavity.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

The present disclosure generally relates to methods for delivering stimulus to mucosal tissue to resurface or remodel the surface of the mucosal tissue. In some embodiments, such resurfacing or remodeling is performed to treat rhinitis. A stimulus, such as for, example, cryoablation spray, may be applied to cause local destruction of the mucosal layer and replacement with healthier mucosal tissue, which may mean one or more of fewer mucous glands, fewer mucous producing cells, less inflammation, less inflammatory cells, thinner mucosal layer or more normal nerve function. While the embodiments discussed herein are generally focused on treating mucosal tissue of the nasal cavity, it should be appreciated that the below-described methods and devices can be used to treat mucosal tissue in other parts of the body, such as the soft palate, gastrointestinal area, or pulmonary area.

In some embodiments, the present disclosure provides systems and methods for applying a cryoablation spray to resurface the mucosal layer. In some embodiments, the stimulus could also be provided by direct RF ablative energy with an algorithm consisting of a plurality of power, voltage, frequency, etc. variables that can be tuned and adjusted depending on the location of the mucosa. Similarly, the stimulus could be delivered via Pulsed Field Ablation, a high voltage, short pulse duration consisting of a pulse train. The pulse duration can be on the order of nanoseconds to milliseconds delivered with a plurality of voltages for the pulse amplitudes and delivered in a pulse train (bursts of packets of pulses). In some embodiments, the treatment could be provided using ultrasonic energy with or without a saline irrigated approach. The saline irrigation could aid in the distribution of the energy.

In some embodiments, the stimulus is delivered to a depth within the mucosal layer sufficient to treat, remodel, or resurface the surface of the mucosal tissue. “Treating,” “remodeling,” or “resurfacing” a tissue, such as a mucosa, as described herein, can specifically relate to changing the properties of the tissue of interest, such as existing mucosal tissue, and replacing the existing mucosal tissue with different or new mucosal tissue. This causes resurfacing of the surface of the mucosal layer. For example, the existing mucosal tissue can be replaced with healthy mucosal tissue. For example, the stimulus can be applied to replace diseased or chronically inflamed mucosal tissue or cells with healthy mucosa that has more normal function. Remodeling or resurfacing the mucosa in the upper airway linings can improve the condition of rhinitis caused by mucus hypersecretion. Treating the tissue with the stimulus can also include ablating the tissue, destroying or shrinking the tissue, or changing cell properties or otherwise causing replacement of the existing tissue with new tissue to resurface the surface of the the mucosal layer. Methods and devices described herein may be used to treat upper airways without the need for more invasive procedures.

In some embodiments, the stimulus may be applied to cause local destruction of the tissue in the mucosal layer and replacement with new mucosal tissue or healthier mucosal tissue. Any treatment, remodeling, or resurfacing of the existing mucosal tissue can result in fewer mucous glands, fewer mucous producing cells, less inflammation, fewer inflammatory cells, thinner mucosal layer, or more normal nerve function. In some embodiments, the application of a stimulus may destroy or modify mucus producing cells. In some embodiments, resurfacing or remodeling the mucosa can include the healing process. For example, in some embodiments, stimulus may be applied to a participant of mucus production in such a way that the healing process causes a change to the shape or function of the one or more physiological participants of mucus production.

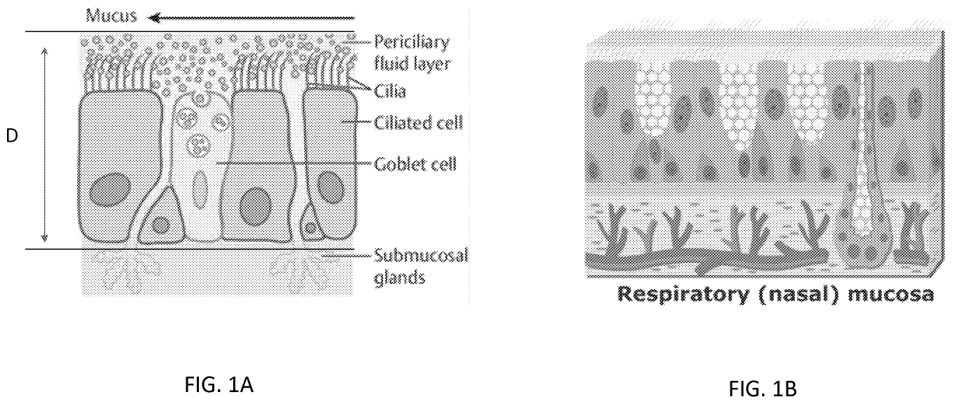

In reference to FIGS. 1A and 1B, the stimulus can be delivered at a desired depth D to resurface the mucosa. In some embodiments, the desired depth D can extend slightly past the mucosa and into the submucosal layer. In some embodiments, the desired depth D may extend to the nerve endings, without disrupting the nerves. In some embodiments, the desired depth may be slightly less than the mucosal layer. In some embodiments, the desired depth may be estimated. For example, the mucosal layer of the nasal cavity is between 0.5 mm and 5.0 mm thick with an average thickness of 3.5 mm. The stimulus can be delivered to approximately 3.5 mm. The location of the mucosa to be resurfaced or remodeled can be at any desirable location. For instance, the mucosa can be in the nasal cavity, over the nasal turbinates, in the superior, middle, or inferior meatus, or in the nasal sinuses, among others. In some embodiments, the present methods and systems are used to vary a function of mucosa, mucosal glands, cilia, goblet cells, nerves, and other tissue. In some embodiments, the stimulus is delivered no further than the nerve endings in the mucosa.

In some embodiments, the stimulus to remodel or resurface the mucosal tissue may be applied in the form of heat, cryotherapy, radiofrequency (RF), laser, light, ultrasound (e.g. high intensity focused ultrasound), microwave energy, electromechanical, mechanical force, cooling, alternating or direct electrical current (DC current), chemical, electrochemical, cryogenic, pulsed field ablation, cryoablation spray, lithotripsy, or others.

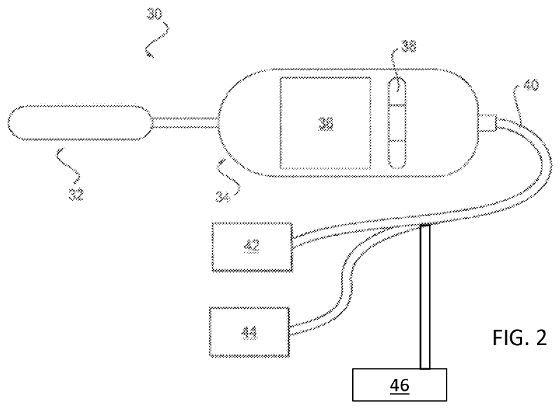

FIG. 2 is a top-view schematic illustration of a mucus reduction treatment device 30, according to one embodiment. The device 30 comprises a treatment element 32 which may be configured to be placed inside the nasal cavity, nasal passage, nasal airway or throat to deliver the desired treatment. In some embodiments, the device 30 may further comprise a handle section 34 which may be sized and configured for easy handheld operation by a clinician. In some embodiments, a display 36 may be provided for displaying information to a clinician during treatment.

In some embodiments, the information provided on the display 36 may include treatment delivery information (e.g. quantitative information describing the stimulus being delivered to the treatment element) or feedback information from sensors within the device or within the treatment element. In some embodiments, the display may provide information on physician selected parameters of treatment, including time, power level, temperature, dispersion rate, volume of a fluid delivered, electric impedance, electric current, depth of treatment or other selectable parameters. In some embodiments, the parameters can be selected based on the particular treatment element 32 of the device 30.

In some embodiments, the handle section 34 may also comprise input controls 38, such as buttons, knobs, dials, touchpad, joystick, etc. In some embodiments, controls may be incorporated into the display, such as by the use of a touch screen. In further embodiments, controls may be located on an auxiliary device which may be configured to communicate with the treatment device 30 via analog or digital signals sent over a cable 40 or wirelessly, such as via BLUETOOTH, WI-FI (or other 802.11 standard wireless protocol), infrared or any other wired or wireless communication method.

In some embodiments the treatment system may comprise an electronic control system 42 configured to control the timing, location, intensity, temperature, amount, or other properties and characteristics of stimulus or other treatment applied to targeted regions of a nasal passageway. In some embodiments, a control system 42 may be integrally incorporated into the handle section 34. Alternatively, the control system 42 may be located in an external device which may be configured to communicate with electronics within the handle section 34. A control system may include a closed-loop control system having any number of sensors, such as thermocouples, electric resistance or impedance sensors, ultrasound transducers, or any other sensors configured to detect treatment variables or other control parameters.

The treatment system may also comprise a power supply 44. In some embodiments, the power supply 44 may be integrally incorporated within the handle section 34. In alternative embodiments, the power supply 44 may be external to the handle section 34. An external power supply 44 may be configured to deliver power to the handle section 34 or the treatment element 32 by a cable or other suitable connection. In some embodiments, the power supply 44 may include a battery or other electrical energy storage or energy generation device. In other embodiments, the power supply 44 may be configured to draw electrical power from a standard wall outlet. In some embodiments, a power supply 44 may also include a system configured for driving a specific stimulus delivery technology in the treatment element 32.

In some embodiments, the power supply 44 may be configured to deliver a low-temperature fluid (e.g. air, water, steam, saline, cryoablation spray, or other gas or liquid) to the treatment element 32 by way of a fluid conduit. For instance, in some embodiments, the treatment system includes a fluid supply 46. In some embodiments, the fluid supply 46 can be a pressurized storage tank fluidly coupled to the device 30 via a fluid supply line. In some embodiments, the fluid supply 46 is a cryogen tank. In some embodiments, the power supply 44 can supply power to drive the supply of fluid from the fluid supply 46 to the device 30, and specifically to the treatment element 32. In some embodiments, the control system 42 can control the application of power from the power supply 44, and in turn control the supply of stimulant (including, in some embodiments, the supply of fluid from the fluid supply 46) to the treatment device 30 and more specifically to the treatment element 32.

In some embodiments, the treatment element 32 may have a substantially rigid or minimally elastic shape and sized and shaped such that it substantially conforms to an ideal shape and size of a patient's nasal passageway or upper airway. In some embodiments, the treatment element 32 may have a curved shape, either concave or convex with respect to the interior of the lateral wall of the nasal passage or upper airway. In some embodiments, the treatment element 32 can be a balloon for delivering cryotherapy. In some embodiments, the treatment element 32 can be a spout or nozzle for delivering cryotherapy.

In some embodiments, the treatment element 32 may be configured to deliver stimulus, such as cryo-therapy, uniformly over an entire outer surface of the treatment element, thereby treating all nasal or upper airway tissues in contact with the treatment element 32. In some embodiments, the treatment element 32 may be configured to deliver stimulus at only selective locations on the outer surface of the treatment element 32 in order to treat selected regions of upper airway tissues. In such embodiments, the treatment element 32 may be configured so that stimulus being delivered to selected regions of the treatment element 32 can be individually controlled. In some embodiments, portions of the treatment element 32 are inert and do not deliver stimulus to the tissue. In some embodiments, the treatment element 32 may be configured with stimulus-delivery (e.g., energy removal) elements distributed over an entire outer surface of the treatment element 32. The control system 42 may be configured to engage such distributed elements individually or in selected groups so as to treat only targeted areas of the upper airway passageway.

In some embodiments, the treatment element 32 may be configured to remove energy from the upper airway tissues. In such embodiments, the treatment element 32 may comprise any suitable cooling element available to the skilled artisan. In some embodiments, the cooling element may comprise conduits for delivering low-temperature fluids onto the tissue. In some embodiments, a low-temperature fluid cooling element may comprise flow channels which place low-temperature fluids into contact with tissues (e.g. through a membrane wall) without injecting such fluids into the patient's nose. In some embodiments, any other suitable cooling element may be provided. In some embodiments, where the treatment element 32 is configured as an energy removal device, the treatment element 32 can be configured to deliver cryogenic therapies configured to transfer heat energy out of selected tissues, thereby lowering the temperature of targeted tissues until a desired level of tissue modification is achieved.

Several embodiments may be employed for delivering stimulus treatment over a desired target area. In some embodiments, the treatment element 32 and control system 42 may be configured to deliver treatment stimulus, such cryotherapy, to a selected tissue depth in order to target treatment at specific tissues. For example, in some embodiments, treatments may be targeted at completely inactivating mucus producing cells. In other embodiments, treatments may be targeted at modifying the mucus production capacity of these cells.

In some embodiments, the treatment element 32 and control system 42 may be configured to deliver treatment stimulus to create specific localized tissue damage, stimulating the body's healing response to create desired conformational or structural changes that reduces the mucus producing ability of the mucus producing cells.

In some embodiments, the treatment device 30 may be configured to cool specific tissue while maintaining a relatively higher temperature in other adjacent tissue. It may be beneficial to confine cryotherapy applied to mainly the tissue to be treated. Nearby tissues such as a basement membrane, submucosal tissue, and skin may be maintained at different temperatures. Limiting unwanted cooling of non-target tissues may allow trauma and pain to be reduced, may reduce scarring, may preserve tissue function, and may also decrease healing time. Combinations of heat transfer or cooling isolation may allow directed treatment of specific tissue such as cells, while excluding another tissue, such as skin, without surgical dissection.

The treatment element 32 may be fixed or adjustable in size. For example, the treatment element 32 may be adjusted to better conform to the upper airway passage of a patient. In the case of a separate positioning element and treatment element 32, a distance between the two elements may either be fixed or adjustable. Adjustability may be accomplished using a variety of means, including, for example, mechanically moving the mold by way of joints, arms, guidewires, balloons, screws, stents, and scissoring arms, among other means. In some embodiments, the mold or repositioning element comprises a balloon configured to reposition or modify tissue. A balloon may also be configured to deliver stimulus, such as cooling using a cryoablation spray.

Specific example embodiments of the treatment device 30 will now be discussed.

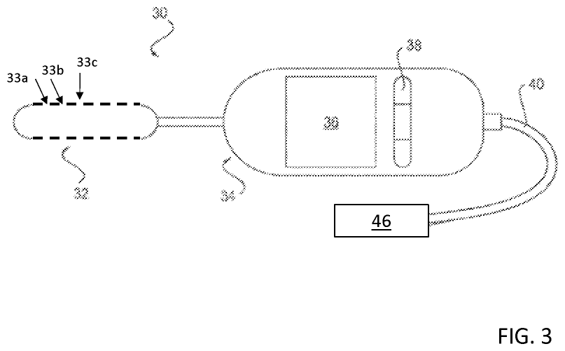

In some embodiments, the treatment device can be a cryotherapy device. In reference to FIG. 3, for instance, in some embodiments, the treatment device 30 includes a catheter with the treatment element 32 at its distal end, where the treatment element 32 is configured to dispense a cryospray through one or more ports 33a, 33b, 33c. The treatment element 32 can be a nozzle, spout, sprayer, or other element configured to dispense a spray or liquid. The treatment device 30, and specifically the treatment element 32, can be fluidly coupled to the fluid supply 46, which can be a pressurized cryogen storage tank. The fluid supply 46 can be fluidly connected (such as, by a supply line 40) and supply the cryospray to the treatment element 32 to dispense the cryospray from the treatment device 30. The cryogen or cryospray, in some embodiments, can be liquid nitrogen. In some embodiments, the treatment device 30 can include a thermocouple for providing temperature feedback of or relating to the target treatment site of the mucosa. By using controls 38 of the handle 34, a clinician can monitor the temperature feedback (for example, on display 36) and adjust one or more variables of stimulant delivery, such as a flow rate of cryospray to and out of the treatment element 32. In some embodiments, the electronic control system 42 can receive the temperature feedback and autonomously adjust one or more variables of stimulant delivery, such as a flow rate of cryospray to and out of the treatment element 32.

In some embodiments (e.g., as shown in FIG. 2) the treatment element 32 may comprise a substantially cylindrical central portion with a semi-spherical or semi-ellipsoid or another shaped end-cap section at proximal or distal ends of the treatment element 32. In some embodiments, the treatment element 32 may comprise a substantially ellipsoid shape as shown.

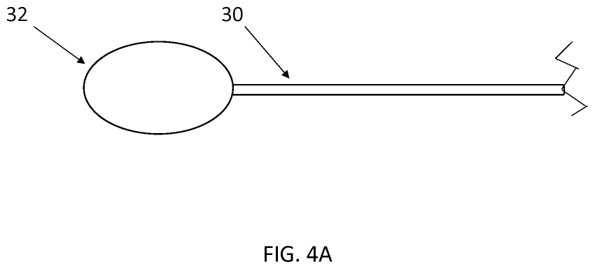

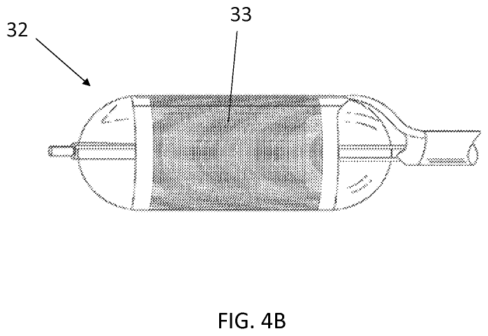

FIG. 4A is a side view of a treatment device 30 having a treatment element 32, according to some embodiments. FIG. 4B is a perspective illustration of a treatment element 32, according to some embodiments. In some embodiments, the treatment element is a balloon that can deliver cryotherapy (e.g., by circulating a low-temperature liquid such as liquid nitrogen through the inflated balloon during treatment). In some embodiments, the balloon can deliver stimulus while it is enlarged to increase the surface area of contact between the treated tissue and treatment element 32. In some embodiments, the treatment element 32 may be an ellipsoid balloon having an asymmetrical shape. In alternative embodiments, the treatment element 32 may have an asymmetrical “egg-shape” with a large-diameter proximal end and a smaller diameter distal end. Any suitable solid or expandable medical balloon material and construction available to the skilled artisan may be used.

In some embodiments, the treatment element 32 can include an expandable balloon that can be inflated with a cryogenic solution. For instance, the balloons can be selectively inflated with the cryogenic solution via a fluidic coupling between the ballon and the fluid supply 46. For instance, the balloon can include an inflation lumen fluidly coupled to the fluid supply 46. The contact between the inflated balloon wall and the mucosa can substantially cool the mucosa to resurface or remodel the mucosa. In such embodiments, the cryogenic solution may not come in direct contact with the mucosa, but can still cool the mucosa through the wall of the inflated balloon. A clinician or the electronic control system 42 can control the volume of inflation of the balloon to best fit the local anatomy of the patient where stimulation is to be delivered, and to remodel or resurface the mucosa as desired. In some embodiments, the balloon can include energy transfer elements 33 positioned on an exterior of the balloon to deliver cooling by circulating a fluid of low temperature though the energy transfer element 33. In some embodiments, a liquid, such as a cryogenic solution can be circulated through the energy transfer elements 33. That is, in some embodiments, the energy transfer elements 33 can be fluidly coupled to the fluid supply 46, which can deliver a cryogenic solution to the energy transfer elements 33. The balloon can also be fluidly coupled to a fluid supply (e.g., liquid or air) which can inflate the balloon to a desired degree to fit the patient's local anatomy. Doing so can place the energy transfer elements 33 in contact with the mucosa to resurface or remodel the mucosa.

Some embodiments of treatment elements 32 may comprise one or more inflatable or expandable sections, such as the balloons described above, configured to expand from a collapsed configuration for insertion into the nasal passageway or throat, to an expanded configuration in which some portion of the treatment element contacts and engages an internal surface of an upper airway or mucosa. In some embodiments, an expandable treatment element 32 may comprise an inflation lumen configured to facilitate injection of an inflation medium into an expandable portion of the treatment element. In some embodiments, an expandable treatment element 32 may comprise one or more segments comprising a shape-memory alloy material which may be configured to expand to a desired size and shape in response to a change of temperature past a transition temperature. In some embodiments, such a temperature change may be brought about by activating a stimulus delivery (e.g., energy-delivery (or removal)) element in the treatment element.

In some embodiments, the treatment element 32 may expand with various locations on the element expanding to different configurations or not expanding at all to achieve a desired shape of the treatment element. In some embodiments, such expandable treatment elements or sections may be elastic, inelastic, or pre-shaped. In some embodiments, expandable treatment elements or sections thereof may be made from shape-memory metals such as nickel-cobalt or nickel-titanium, shape memory polymers, biodegradable polymers or other metals or polymers. Expandable balloon elements may be made of any elastic or inelastic expandable balloon material.

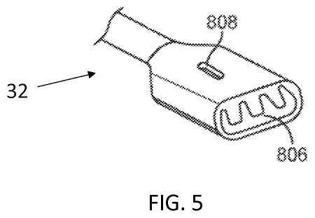

In some embodiments, as shown in FIG. 5, the treatment element 32 can include a cooling member 806 and an air vent 808. In such embodiments, a vacuum source can be coupled to the treatment device 30 in the same manner as the fluid supply 46 discussed above. That is the vacuum source can be fluidly coupled to the treatment device 30, and particularly the treatment element 32, to supply vacuum pressure at the treatment element 32. It should be appreciated that the vacuum source can be coupled to the treatment device 30 in addition to the fluid supply 46. A clinician can manually, or the electronic control unit 42 can autonomously, control the vacuum source to create a desired suction at the treatment element 32. Specifically, operation of the vacuum source can cause the cooling member 806 to deliver suction at the extreme distal end of the treatment element 32, thus sucking air into the cooling member 806 and proximally into the treatment device 30. In addition, some air may pass through the air vent 808 on the top of the treatment element 32. The passage of air through the cooling member 806 causes convection cooling inside the cooling member 806, which in turn applies conduction cooling to the mucosa that is in contact with the cooling member 806. In various embodiments, the extreme distal end of the treatment element 32 may have one or more openings leading into the cooling member, may include a filter of the opening(s), or the like. The tissue contact surface of the cooling member 806 may be made of metal, plastic, polymer or any suitable material so as to convey the cooling to the mucosa. In some embodiments, different types of cooling members 806 may be used, such as but not limited to water circulating cooling members, cryogenic cooling members and the like.

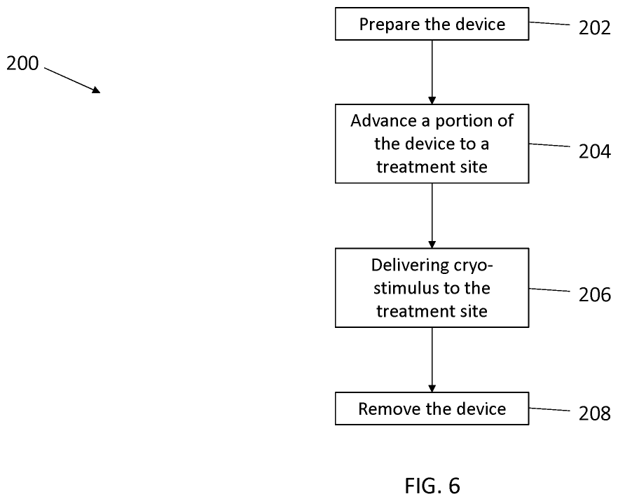

FIG. 6 is a flow diagram of an example method 200 of use of various devices, including the device 30. In certain implementations, the method 200 may include preparing the device, such as the device 30, at a step 202, advancing a portion of the device to a treatment site at a step 204, delivering stimulus to the treatment site at a step 206, and removing the treatment device at a step 208.

In some embodiments, the method 200 may start at the step 202 of preparing the device, such as the treatment device 30. Preparing the device may include removing the device from sterile packaging, assembling one or more components of the device, sterilizing the device, attaching the device to an energy source, or other preparatory work. In certain implementations, this step may include customizing the device to suit the particular needs of the patient and the clinician. This may include articulating, manipulating, or otherwise changing one or more components of the device. For example, a clinician may articulate or bend a shaft or the treatment portion to place the device in a first, desired configuration. The first, desired configuration may be selected to facilitate navigation of the nasal anatomy of the particular patient to reach the desired treatment site.

In some embodiments, preparing the device at step 202 may follow removing the treatment device from the patient. For example, the clinician may determine that the device is not suitable in its current state, remove the device, and then reconfigure the device in a more suitable format (e.g. a second configuration) in step 202.

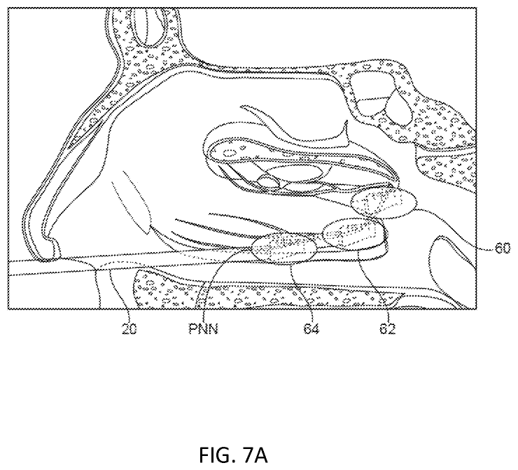

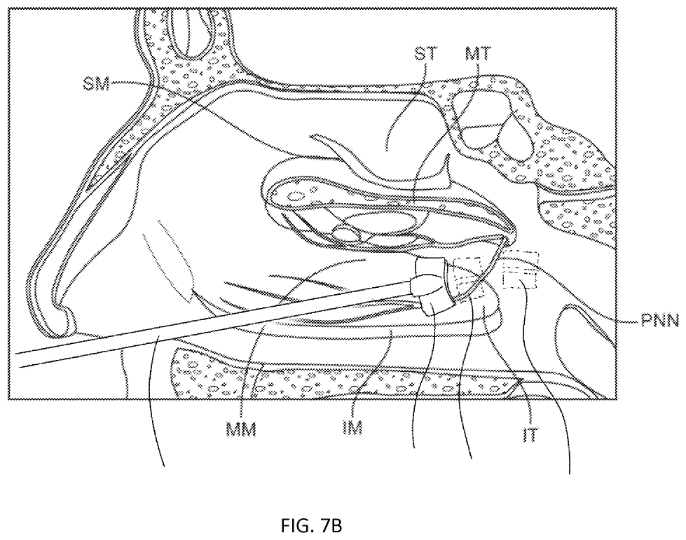

As shown in FIG. 7A, advancing a portion of the device to a treatment site at step 204 may follow the preparation of the device at step 202. For example, the clinician may navigate the patient's nasal anatomy with the device in the desired configuration. Specifically, the clinician may advance a treatment portion of the treatment device into a nostril of the patient to contact a treatment surface of the treatment portion with mucosal tissue of the upper airway. For example, the site of mucosal treatment can be over the nasal turbinates, in the superior, middle, or inferior meatus, or in the nasal sinuses, among others. This step may be performed without piercing the mucosal tissue. The device is advanced to be to place the treatment element in contact with the treatment site.

FIG. 7B shows an exemplary embodiment of multiple treatments applied to target tissue with a multichannel device. Pairs of dashed rectangles show previous treatments.

During navigation, the clinician may perform one or more tests to determine whether proper contact with the treatment site has been made. In some embodiments, the clinician may activate one or more pairs of the electrodes on the treatment device. Based on measured results, the clinician may determine that proper contact has not been achieved because an energy pathway could not be made between one or more pairs of electrodes or that one or more measured electrical parameters (e.g., impedance, voltage, current, temperature, etc.) is outside of a desired range. In some embodiments, the clinician may attempt to apply pressure to the treatment site with the treatment portion and determine by feel whether proper contact has been made. In some embodiments, clinician may take a reading using a thermocouple or an electrode to determine whether proper contact has been made. Based on the one or more tests, the clinician may determine that proper contact has been made between the treatment portion and the treatment site. During navigation, a navigation system may be used to position the device based on previously obtained images.

Based on the one or more tests, the clinician may determine that proper contact has been made between the treatment portion and the treatment site. In this situation, the method may move to the step 206 of delivering stimulus (e.g., a therapy) to the treatment site. In certain circumstances, the clinician may determine that proper contact has not been made or that the device is otherwise unsuitable in its current state. In this situation, the method may move to the step 208 of removing the treatment device.

In the step 206 of delivering stimulus to the treatment site, the clinician may cause the device to apply stimulus to the treatment site. For example, in certain implementations, a clinician may use the device to apply a cryotherapy to the posterior aspect of the inferior turbinate. For this treatment, it may be desirable to press the treatment portion against the tissue of the posterior aspect of the inferior turbinate such that the tissue substantially conforms to the shape of the treatment element.

When proper contact is made at the treatment site, the treatment element, such as the treatment element 32, can be activated to deliver therapy stimulus to the treatment site. Merely as an example, the clinician can provide a cryotherapy to the treatment site using any one of the above-discussed treatment devices 30. The stimulus can be any of the above-described cryotherapy or cooling stimuli and can be delivered to achieve a desired resurfacing or remodeling of the mucosal tissue. Variables related to the stimulus can be modified before and during stimulus delivery to remodel or resurface the tissue at the treatment site as desired. For instance, a cryospray temperature, a volume of cryospray, or other variable can be controlled for selective remodeling and resurfacing and based on the location of the mucosa being treated.

Delivery of the stimulus can remodel or resurface the mucosa such that the diseased or chronically inflamed mucosal cells are replaced with healthy mucosa that have more normal function, such as producing less mucus or other fluids, being less prone to allergy, and having less nerve stimulation or less activity of inflammatory cells. This can be through modification of the cell types that comprise the mucosal layers. For example, this can include removing nerve ending present in the mucosal layer. Delivery of the stimulus can remodel or resurface the mucosa such that the mucosal layer would be locally destroyed and replaced with healthier mucosal tissue (which may mean fewer mucous glands, mucous producing cells, less inflammation, or more normal nerve function).

As is shown in FIG. 7A, the physician may deliver energy at one location and then move the treatment portion to another treatment site and repeat the steps of confirming contact and delivering energy. For example, as shown in FIG. 7A, the treatment sites include a first target location 60, a second target location 62 and a third target location 64, all of which fall along the path of a posterior nasal nerve PNN. Additional treatments may optionally be provided in the same general area or in different areas of the nasal cavity. In certain circumstances, the clinician may determine that proper contact has not been made and the physician can then either re-orient the treatment portion or move it to another treatment site.

Delivery of the stimulus can be monitored during delivery. For instance, the treatment device can be equipped to monitor temperature of the treated tissue or adjacent tissues or monitor the impedance of the tissues in contact with the device to monitor for changes to the treated or nearby tissue, to execute a tissue apposition check, or monitor other biometric information. Based on the feedback, one or more variables relating to stimulus delivery can be adjusted to achieve a desired resurfacing or remodeling of the mucosa. In some embodiments, the feedback can indicate when a treatment is complete and result in the cessation of stimulus delivery. The feedback can be presented to and analyzed by the clinician (e.g., on the display 36), who can then manually adjust the stimulus delivery variables (e.g., via the input controls 38). In some embodiments, a controller, such as the electronic control system 42, can autonomously analyze the feedback and adjust the stimulus delivery variables to achieve a desired remodeling or resurfacing of the mucosa.

The step 208 of removing the treatment device can follow delivery of stimulus to the treatment site. In certain circumstances, the clinician may remove part or all of the device from the nasal anatomy of the patient. The clinician may determine that one or more further adjustments may improve contact between the treatment portion and the treatment site or otherwise achieve improved therapeutic results. In such circumstances, the method may return to the step 202 of preparing the device. For example, the clinician may articulate one or more components of the device to place the device in a second configuration. The clinician may then navigate the nasal anatomy again and deliver stimulus to the treatment site.

When the step 208 of removing the treatment device follows delivering stimulus to the treatment site, the clinician may simply remove the device from the patient and end the procedure. In certain other circumstances, the clinician may partially or wholly remove the device and repeat one or more of the steps of the method 200 in order to more fully treat the treatment site or treat other treatment sites. In certain circumstances, the method 200 may end after step 208. While this method 200 has been described with reference to the device 30, it should be appreciated that the method 200 may be used with some or all of the other devices and methods disclosed herein.

The steps above and much of the disclosure is described with respect to the nose and nasal cavity, but it will be understood that the systems, devices, and methods described herein can be used in other areas of the body, including but not limited to the mouth, including the soft palate and the base of the tongue, the throat, including nerves in the throat, and the ear canal.

In some embodiments, energy used by the systems and devices described herein may be applied in the form of heat, radiofrequency (RF), pulsed radiofrequency, laser, light, ultrasound (e.g., high intensity focused ultrasound), microwave energy, electromechanical, mechanical force, cooling, alternating or direct electrical current (DC current), chemical, electrochemical, cryotherapy, cryogenic, pulsed field ablation, cryoablation spray, lithotripsy, histotripsy, shock wave, or others. In some embodiments, the nasal valve or surrounding tissues may be strengthened through the application of cryogenic therapy, or through the injection or application of bulking agents, glues, polymers, collagen or other allogenic or autogenic tissues, or growth agents.

While the above discussion has particularly focused on the use of the treatment device 30 as an energy removal device to deliver cryotherapy, it should be appreciated that the treatment device 30 described above can, in some embodiments, deliver any other stimulation for treatment. For instance, the treatment element 32 can be configured to deliver energy to the mucosa to remodel or resurface the mucosa.

For instance, in some embodiments, the power supply 44 may be configured to deliver a radio frequency alternating current signal to an RF energy delivery element. In some embodiments, the power supply 44 may be configured to deliver a signal suitable for delivering ultrasound or microwave energy via suitable transducers. In some embodiments, the power supply 44 may be configured to deliver a high-temperature fluid (e.g. air, water, steam, saline, cryoablation spray, or other gas or liquid) to the treatment element 32 by way of a fluid conduit. For instance, in some embodiments, the treatment system includes the fluid supply 46, and the power supply 44 can supply power to drive the supply of fluid from the fluid supply 46 to the device 30, and specifically to the treatment element 32. As noted above, in some embodiments, the control system 42 can control the application of power from the power supply 44, and in turn control the supply of stimulant to the treatment device 30 and more specifically to the treatment element 32. In some embodiments, the treatment element 32 may be configured to deliver stimulus in the form of energy (e.g. heat, RF, ultrasound, microwave).

In some embodiments, the treatment element 32 may be configured to deliver heat energy to the upper airway tissues. In such embodiments, the treatment element 32 may comprise any suitable heating element available to the skilled artisan. For example, the treatment element 32 may comprise electrical resistance heating elements. In alternative embodiments, the heating element may comprise conduits for delivering high-temperature fluids (e.g. hot water or steam) onto the tissue. In some embodiments, a high-temperature fluid heating element may comprise flow channels which place high-temperature fluids into conductive contact with tissues (e.g. through a membrane wall) without injecting such fluids into the patient's nose. In some embodiments, any other suitable heating element may be provided. In further embodiments, the treatment element 32 may comprise elements for delivering stimulus in other forms such as light, laser, RF, microwave, cryogenic cooling, DC current or ultrasound in addition to or in place of heating elements. In some embodiments, the treatment element 32 may be an inflatable balloon with energy delivery elements that deliver heat by circulating a fluid of elevated temperature though the inflated balloon during treatment. In some embodiments, the balloon can deliver stimulus while it is enlarged to increase the surface area of contact between the treated tissue and treatment element 32.

Several embodiments may be employed for delivering stimulus treatment over a desired target area. For example, in some embodiments, a laser treatment system may treat a large surface area by scanning a desired treatment pattern over an area to be treated. In the case of microwave or ultrasound treatment, suitably configured transducers may be positioned adjacent to a target area and desired transducer elements may be activated under suitable depth focus and power controls to treat a desired tissue depth and region of the mucosa. In some embodiments, ultrasound or microwave treatment devices may also make use of lenses or other beam shaping of focusing devices or controls. In some embodiments, one or more electrical resistance heating elements may be positioned adjacent to a target region and activated at a desired power level for a therapeutically effective duration. In some embodiments, such heating elements may be operated in a cyclical fashion to repeatedly heat and cool a target tissue. In some embodiments, RF electrodes may be positioned adjacent to and in contact with a targeted tissue region of the mucosa. The RF electrodes may then be activated at a frequency and power level for a therapeutically effective duration. In some embodiments, the depth of treatment may be controlled by controlling a spacing between electrodes. In some embodiments, RF electrodes may include needles which may puncture a nasal or upper airway tissue (e.g., the mucosa) to a desired depth.

In some embodiments, the treatment element 32 and control system 42 may be configured to create specific localized tissue damage without the application of energy. For example, the treatment element 32 may be configured to chemically cauterize tissue in an upper airway passage by delivering a cauterizing agent (e.g., silver nitrate, trichloroacetic acid, cantharidin, etc.) to the tissue. The treatment element 32 may comprise apertures configured to permit the cauterizing agent pass through to the upper airway. In some embodiments, the treatment element 32 may aerosolize the cauterizing agent. Other delivery methods are also contemplated. The treatment element 32 may comprise a lumen through which the cauterizing agent passes. The lumen may be fluidly connected to a reservoir or container holding the cauterizing agent. The device may comprise an input control (e.g., a button or switch) configured to control the delivery of the cauterizing agent. In some embodiments, the treatment element 32 comprises an applicator that can be coated in a cauterizing agent (e.g., dipped in a reservoir of cauterizing agent, swabbed with cauterizing agent, etc.) and the coated treatment element applicator may be applied to tissue to be treated. In some embodiments, the treatment element 32 may be configured to apply cauterizing agent to the patient over a prolonged period of time (e.g., 30 seconds, 1 minute, 2 minutes, etc.). In some embodiments, the treatment element 32 comprises shields configured to protect tissue surrounding the tissue to be treated from coming into contact with the cauterizing agent. In some embodiments, a separate element is used to shield tissue surrounding the tissue to be treated from coming into contact with the cauterizing agent. While such treatments may be performed without the application of energy, in some embodiments, they are performed in conjunction with energy or cryotherapy treatments.

In some embodiments, the treatment device 30 may be configured to treat specific tissue while maintaining a relatively lower temperature in other adjacent tissue. It may be beneficial to confine heat energy applied to mainly the tissue to be treated. Nearby tissues such as a basement membrane, submucosal tissue, and skin may be maintained at different temperatures. Limiting unwanted heating of non-target tissues may allow trauma and pain to be reduced, may reduce scarring, may preserve tissue function, and may also decrease healing time. Combinations of heat transfer or heat isolation may allow directed treatment of specific tissue such as cells, while excluding another tissue, such as skin, without surgical dissection.

In some embodiments, the mold or repositioning element of the device 30 comprises the balloon configured to reposition or modify tissue. In some embodiments, the balloon may also be configured to deliver stimulus, such as heat using hot liquid or gas.

In some embodiments, the energy transfer elements 33 positioned on the exterior of the balloon can be configured to deliver energy to the mucosa or other tissue the balloon positioned the energy transfer elements 33 in contact with. For instance, in some embodiments, the energy transfer elements 33 can be configured to deliver heat to remodel or resurface the mucosa.

It should be appreciated that the method 200 discussed above can be carried out with any treatment device 30, including any treatment element 32, discussed herein. For instance, the method 200 can be carried out with a treatment device 30 configured to deliver stimulus in the form of energy (e.g., heat) to remodel or resurface the mucosa. Merely as examples, the clinician can activate one or more pairs of electrodes of the treatment element 32 to deliver stimulus to the treatment site. The stimulus can be any of the above-described stimuli and can be delivered to achieve a desired resurfacing or remodeling of the mucosal tissue. As described above, variables related to the stimulus can be modified before and during stimulus delivery to remodel or resurface the tissue at the treatment site as desired. In some embodiments, for instance, utilizing RF ablation, the power, voltage, or frequency of energy, or other variable can be controlled for selective remodeling and resurfacing and based on the location of the mucosa being treated. In some embodiments, for instance, utilizing pulsed field ablation, the pulse durations, voltage amplitudes of each pulse, or other variable can be controlled for selective remodeling and resurfacing and based on the location of the mucosa being treated. In some embodiments, for instance utilizing ultrasonic energy, amplitude, frequency, treatment length, pulsation or continuity, or other variable can be controlled for selective remodeling and resurfacing and based on the location of the mucosa being treated. The ultrasonic energy could be delivered with or without a saline irrigation, which can also be selectively controlled in its volume of delivery to aid in distribution of energy and to selectively remodel and resurface the mucosa being treated.

The systems and methods of the present disclosure can be used to treat various conditions inside or outside of the nasal cavity. It should be appreciated that, while the devices and methods described herein are described in relation to a nasal cavity, other areas of the body, including the mouth and ear, can be treated. Some of the conditions that may be treated by the embodiments described herein include, but are not limited to, rhinitis, asthma, COPD, emphysema, airway inflammation, nasal obstruction or congestion, eye inflammation and allergic conjunctivitis, chronic cough, post nasal drip, allergies, nasal polyps, sinusitis, turbinate enlargement, migraine headaches, other headaches (e.g., cluster headaches, nasal contact point headaches, etc.), tinnitus, dizziness, vertigo, dry eye, excessive tearing, empty nose syndrome, pain (e.g., facial nerve pain, trigeminal neuralgia, complex regional pain syndrome, etc.), sleep apnea, snoring, disordered breathing during sleep, anxiety, mood disorders, middle ear conditions (e.g., otitis media), Eustachian tube dysfunction, herpes zoster, paroxysmal hemicranias, cancer of the head or neck, and reduction of chemical mediators that lead to any of the above-listed conditions. It should be noted that many of these conditions may be co-morbid with one another, and so a treatment of one condition may alleviate one or more other conditions.

By way of non-limiting example, in some embodiments, the systems and methods of the present disclosure can be used to treat or alleviate rhinitis, which can be generally described as the inflammation of the nasal mucosa due to allergic or non-allergic triggers, which leads to excessive mucus production in the nasal cavity. This increased mucus can cause runny nose (rhinorrhea), post nasal drip syndrome (PNDS), upper airway cough syndrome (UACS), congestion, itching, and sneezing symptoms.

In some embodiments, the systems and methods of the present disclosure can be used to decrease mucus production in the upper airways. Mucus hypersecretion often occurs when a mucus producing cell displays rapid mucus secretion behavior. In some embodiments, to prevent this, properties of the cell can be modified to inactivate or retard the cell so as to halt or impede the release of mucins into the airway. In some embodiments, modifying the properties of the mucus producing cells may include reducing the rate at which cells produce mucus. In some embodiments, modifying the properties of mucus producing cells may include deactivating some or all mucus producing cells in a region. In some embodiments, the cells can be modified by being locally destroyed and can be replaced with healthier mucosal tissue, which may mean fewer mucous glands, fewer mucus producing cells, less inflammation, or more normal nerve function. While some embodiments may change a property of a tissue, such as an ability of a tissue to produce mucus, other embodiments may change a tissue from one type to another type. One example of this latter change is changing mucosal tissue containing goblet cells to mucosa with fewer goblet cells or scar tissue. These various embodiments may be used to reduce movement of mucus, reduce the amount of mucus produced, reduce frequency of mucus production, change the mucus viscosity/consistency, or change the path of mucus flow. In some embodiments, the systems and methods of the present disclosure can be used to apply energy to stimulate or disrupt various nasal nerves (as described below) to decrease mucus production in the upper airways.

By way of another non-limiting example, in some embodiments, the systems and methods of the present disclosure can be used to treat or alleviate migraine or headaches, such as cluster headaches, nasal contact point headaches, tension headaches, sinus headaches, or similar conditions. In some embodiments, the systems and methods of the present disclosure can be used to apply energy to stimulate or disrupt various nasal nerves (as described below) to alleviate migraines or headaches.

By way of another non-limiting example, in some embodiments, the systems and methods of the present disclosure can be used to treat or alleviate sleep apnea, disordered breathing during sleep other sleep disorders. In some embodiments, tissues, such as mucosa, of the soft palate in the mouth can be treated, resurfaced, or altered. This change in the soft palate can prevent collapse of the soft palate or vibration of the soft palate during nighttime breathing and thus prevent or at least reduce sleep apnea or snoring. One example of this is reducing nasal congestion through treatment of the nasal nerves leading to better breathing during sleep. This also reduces the negative pressure generated in the nasal cavity on inspiration leading to less airway collapse.

In some embodiments, the systems and methods of the present disclosure used to apply a stimulus to cause mucosal remodeling may also neuromodulate certain nerves within the nasal cavity. In some embodiments, any nerve or combination of nerves in the nasal cavity can be treated to treat various conditions inside or outside of the nasal cavity. Generally, nasal neuromodulation may involve treating one or more nerves to change or disrupt their functions without completely stopping their function. At the same time, however, nasal neuromodulation may involve completely destroying the nerve tissue or stopping the function of one or more nerves while allowing one or more other nerves in the same area to maintain their function. Typically, the systems of the present disclosure will contact the mucosa overlying one or more of these one or more nerves, and energy will be delivered to resurface mucosa, which may also neuromodulate the nerves. In some embodiments, the treatments of the present disclosure may involve disruption of parasympathetic or sensory nerve signals in one or more nerves (e.g., the posterior nasal nerve). In some embodiments, the resurfacing of the mucosal layer may also impact the submucosal tissue, as discussed above, and also change or disrupt the functions of the nerves in the same region (e.g., the posterior nasal region).

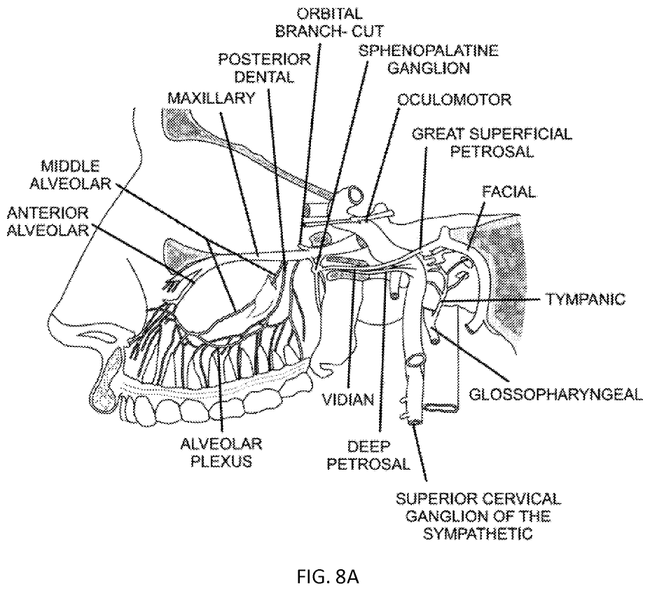

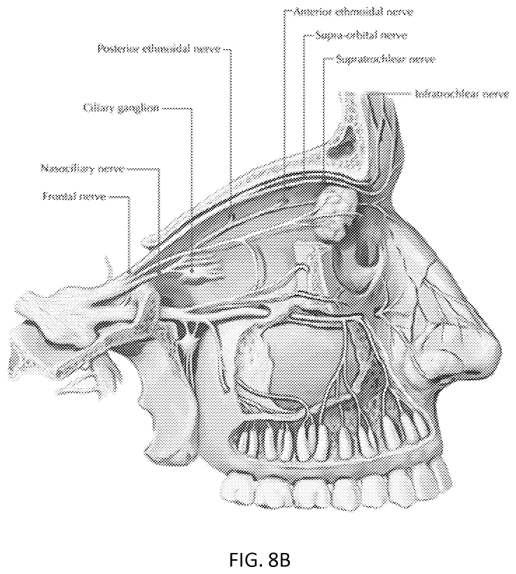

FIGS. 8A and 8B are a sagittal views of a human nasal cavity, illustrating the lateral wall and nasal nerves that are related to the treatment of mucosal tissue as described herein. Referring to FIG. 8A, a sagittal cross-section of a human nasal cavity is shown. In this cross-sectional view, bone has been removed to illustrate the path of multiple different nerves, such as the vidian nerve and the sphenopalatine ganglion. According to various embodiments and examples described herein, any nerve or combination of nerves may be treated, such as but not limited, to the vidian nerve, vidian nerve branches, the sphenopalatine ganglion or any nerve branching from the sphenopalatine ganglion, such as the posterior nasal nerves (PNN), the posterolateral nasal nerves (PLNN), the posterior inferior lateral nasal nerves, and the posterior superior lateral nasal nerves (descending from the sphenopalatine ganglion in FIG. 8A but not labeled).

In reference to FIG. 8B, in some embodiments, nerves in the nasal cavity that can be affected or treated include, but are not limited to, the frontal nerve, nasociliary nerve, ciliary ganglion, posterior nasal nerve, posterior ethmoidal nerve, supra-orbital nerve, infratrochlear nerve, olfactory nerve, trigeminal nerve, nasopalatine nerve, maxillary nerve, sphenopalatine ganglion, anterior nasal nerves, and the anterior ethmoidal nerve and its branches.

In some embodiments, the present disclosure includes neuromodulation of nerves in the posterior nasal region, such as posterior nasal nerves (PNN), the posterolateral nasal nerves (PLNN), the posterior inferior lateral nasal nerves, and the posterior superior lateral nasal nerves.

Embodiments have been described where the techniques are implemented in circuitry or computer-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

While embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments described herein can be employed. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification, embodiments have been described in a way which enables a clear and concise specification to be written, but it is intended and will be appreciated that embodiments may be variously combined or separated without parting from the scope of the present disclosure. It is intended that the present disclosure be limited only to the extent required by the appended claims and the applicable rules of law.

As utilized herein, the terms “comprise” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art. The use of the terminology X “or” Y herein should be interpreted as meaning either “X” or “Y” individually, or both “X and Y” together.

All patents and publications mentioned in this specification are herein incorporated by reference to the same extent as if each independent patent and publication was specifically and individually indicated to be incorporated by reference.

It is also to be understood that the following claims are to cover all generic and specific features of the disclosure described herein, and all statements of the scope of the disclosure which, as a matter of language, might be said to fall therebetween.

What is claimed is:

1. A method of resurfacing a mucosal layer, the method comprising:
applying a stimulus to an existing mucosal tissue to a predetermined depth, the predetermined depth being a depth within a mucosal layer,
wherein the stimulus treats the existing mucosal tissue along the predetermined depth to cause replacement of the existing mucosal tissue with new mucosal tissue, thereby resurfacing a surface of the mucosal layer.

2. The method of claim 1, wherein the predetermined depth extends to a distal end of nerve endings in the existing mucosal tissue.

3. The method of claim 1, wherein the predetermined depth is less than a depth of the mucosal layer.

4. The method of claim 1, wherein the predetermined depth is 3.5 mm.

5. The method of claim 1, wherein the stimulus comprises a cryoablation spray.

6. The method of claim 1, wherein the stimulus is radiofrequency energy.

7. The method of claim 1, wherein the stimulus is pulsed field ablation.

8. The method of claim 1, wherein the resurfacing of the surface of the existing mucosal tissue causes fewer mucous glands, fewer mucous producing cells, less inflammation, less inflammatory cells, thinner mucosal layer, or more normal nerve function.

9. A method of resurfacing a mucosal layer, the method comprising:
advancing a treatment device into a nasal cavity;
controlling, with a control system, a depth and amount of a stimulus being applied to mucosal tissue in the nasal cavity;
applying the stimulus with the treatment device to the mucosal tissue to a depth selected by the control system, wherein the depth is selected allow the stimulus to treat the mucosal tissue to resurface a surface of the mucosal tissue; and
removing the treatment device from the nasal cavity.

10. The method of claim 9, wherein the stimulus treats the mucosal tissue along the depth to cause replacement of the mucosal tissue with healthy mucosal tissue, thereby resurfacing a surface of the mucosal layer.

11. The method of claim 9, wherein the depth extends to a distal end of nerve endings in the mucosal tissue.

12. The method of claim 9, wherein the depth is less than a depth of the mucosal layer.

13. The method of claim 9, wherein the depth is 3.5 mm.

14. A method of resurfacing a mucosal layer, the method comprising:
advancing a treatment device into a nasal cavity, the treatment device comprising:
a treatment element having one or more ports for delivering a stimulus in the form of a cryoablation spray;
a stimulus supply in fluid communication with the one or more ports of the treatment element,
a control system configured to control an amount of stimulus delivered from the treatment element;
applying power, with a power supply, to drive an amount of the stimulus from the stimulus supply to the treatment element to apply the stimulus to treat existing mucosal tissue along a predetermined depth, the predetermined depth being a depth within a mucosal layer,
wherein the predetermined depth is selected to allow the stimulus treat the existing mucosal tissue along the predetermined depth to cause replacement of the existing mucosal tissue with healthy mucosal tissue, thereby resurfacing a surface of the mucosal layer; and
removing the treatment device from the nasal cavity.

15. The method of claim 14, wherein the resurfacing of the surface of the existing mucosal tissue causes fewer mucous glands, fewer mucous producing cells, less inflammation, less inflammatory cells, thinner mucosal layer, or more normal nerve function.

16. The method of claim 14, wherein applying the stimulus causes one or more changes to properties of the mucosal layer.

17. The method of claim 14, wherein the predetermined depth extends to a distal end of nerve endings in the mucosal layer, without disrupting the nerves.

18. The method of claim 14, wherein the predetermined depth is less than a depth of the mucosal layer.

19. The method of claim 14, wherein the predetermined depth is 3.5 mm.

20. The method of claim 14, wherein the treatment element includes one or more thermocouples for providing temperature feedback of or relating to the mucosal tissue, the temperature feedback allowing for adjusting one or more variables of delivery of the cryoablation spray, including a flow rate of cryoablation spray to the one or more ports of the treatment element or a flow rate of the cryoablation spray out of the one or more ports of the treatment element.
